(19) United States
(12) Patent Application Publication
(10) Pub. No.: US 2005/0136464 A1
(43) Pub. Date: Jun. 23, 2005

(54) Quantitation of biomolecule in a complex mixture by serial combinatorial dilution

(76) Inventors: Peter Berndt, Basel (CH); Stefan Evers, Muellheim (DE); Hanno Langen, Steinen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/016,588

(22) Filed: Dec. 17, 2004

(30) Foreign Application Priority Data
Dec. 18, 2003 (EP) 03104775.6

(51) Int. Cl. C12Q 1/68; G01N 33/53

(52) U.S. Cl. 435/6; 435/7.1; 702/19

(57) ABSTRACT

The invention provides a method for the quantification of a biomolecule in a complex mixture of biomolecules which comprises a fractionation of the mixture of biomolecules providing at least two fractions with at least one distinct component each. These fractions are then subjected to serial combinatorial dilution. Subsequently, the biomolecule is detected and identified in the fractions by a method providing a sensitivity threshold and identify information. The quantity of the biomolecule is determined by summarizing the number of identifications of the biomolecule in each fraction on each dilution level in consideration of the respective dilution factor. For purpose of normalization this sum may be divided by the total number of identifications of all biomolecules in all fractions on all dilution levels.

### BACKGROUND OF THE INVENTION

A current method for detection of a biomolecule (for example a protein) are the two dimensional gel electrophoresis with subsequent volumetric analysis of the stained gel. However, it is difficult to determine the quantity of the analyzed biomolecule, especially if its quantities in different samples shall be compared. To account for the inter-sample variation in biomolecule concentrations ti)e gels have to be processed in parallel and a gel-to-gel-matching has to be done.

Additionally, for realistic samples in proteomics, methods described in the art have limited applicability. Gel comparison is only realistic for small series of very similar samples. Because of limitations of the analytical process, gel matching is very hard to automate and ultimately involves human operator input. The number of comparisons to make is proportional to the square of the number of gels, which limits the method to sets of a few tens of gels. Parallel processing involves either the isotopic or bacterial cultures, or small model organisms. Chemical modifications have limited penetration (not all of the sample will be modified or the modification might not be detectable for all labeled molecules) and must be chosen extremely carefully in order to not interfere with the separation process. In both cases, combination of the sample with a control is required to obtain a reliable measurement, which can present a problem when controls are scarce (e.g., healthy human tissues), or not available at the time the sample is processed.

There is a need of a simpler method for quantification of biomolecules in a complex mixture of biomolecules. The method of the present invention is simpler, easier and better to apply than the methods of prior art. Additionally, the method of the present invention is generally cheaper to perform than the methods described in the prior art. In many realistic cases, the method of the present invention will be the only method that can be applied to simply and easily quantify a biomolecule.

### SUMMARY OF THE INVENTION

The present invention relates to a method for the quantification of a biomolecule in a complex mixture of biomolecules comprising fractionation of the complex mixture into fractions with subsequent serial combinatorial dilution of the fractions and detection of the biomolecules in each original fraction and each diluted fraction by a method with a defined sensitivity threshold and identification capabilities.

The present invention provides a method for the quantification of a biomolecule in a complex mixture of biomolecules comprising
a. providing at least two fractions derived from the fractionation of the complex mixture of biomolecules comprising each at least one distinct biomolecule component,
b. subjecting the fractions to a serial combinatorial dilution step,
c. detecting and identifying the biomolecule in each original fraction and each diluted fraction by a method with a stable and well defined sensitivity threshold and identity information, and
d. quantifying the biomolecule in the complex mixture of biomolecules by summarizing the number of identifications of the biomolecule in each fraction on each dilution level in consideration of the respective dilution factor.

For the purpose of normalization the sum of d) may be divided by the total number of identifications of all biomolecules in all fractions on all dilution levels (dilution levels of original fractions and diluted fractions).

The method of the present invention for the quantification of a biomolecule provides a relative quantification of one or more biomolecules in a complex mixture of biomolecules from one source compared to the respective biomolecules in a complex mixtures from other sources.

This method is independent of the properties of the various biomolecules. Polynucleotides, polypeptides or carbohydrates, as well as other biomolecules, may be processed by the method of the invention. A further advantage of this method is that it combines quantification with the identification of a biomolecule in a simple manner without the need for additional efforts targeted at biomolecule quantitation. Moreover, if the quantity of a biomolecule derived of one source shall be compared with the one of another source the mixtures of biomolecules may processed separately of each other.

### BRIEF DESCRIPTION OF THE FIGURES

FIG. 1 illustrates an example of the method of the present invention: In a first step the complex mixture is fractionated into different fractions. These fractions are then subjected to a serial combinatorial dilution. In a second step a biomolecule is detected by for example two dimensional gel electrophoresis on the sample pools with subsequent mass spectrometric identification.(AU: Absorption Unit; 8 to 23: Fractions)

FIG. 2 shows the calculation of the relative quantity of a biomolecule. q: relative quantity of a biomolecule; Nᵢ: the number N of identifications of an individual biomolecule on dilution level i; dᵢ: the dilution factor d of the respective dilution level i; Nₜₒₜₐₗ: the total number N of identifications of all biomolecules in all fractions on all dilution levels. (Scheme: N1: undiluted, N2: 2-fold dilution, N3: 4-fold dilution, N4: 8-fold dilution)

FIG. 3 shows the number of identifications for the proteins glycogen phosphorylase (a), vimentin (b)and the heat shock protein 105 (c) in two dimensional electrophoresis gels from level 1 (no dilution), level 2 (2-fold dilution), level 3 (4-fold dilution), and level 4 (8-fold dilution). The values were added up from experiments carried out in triplicate. (Control: 5 mM Glucose; high Glucose: 10 mM)

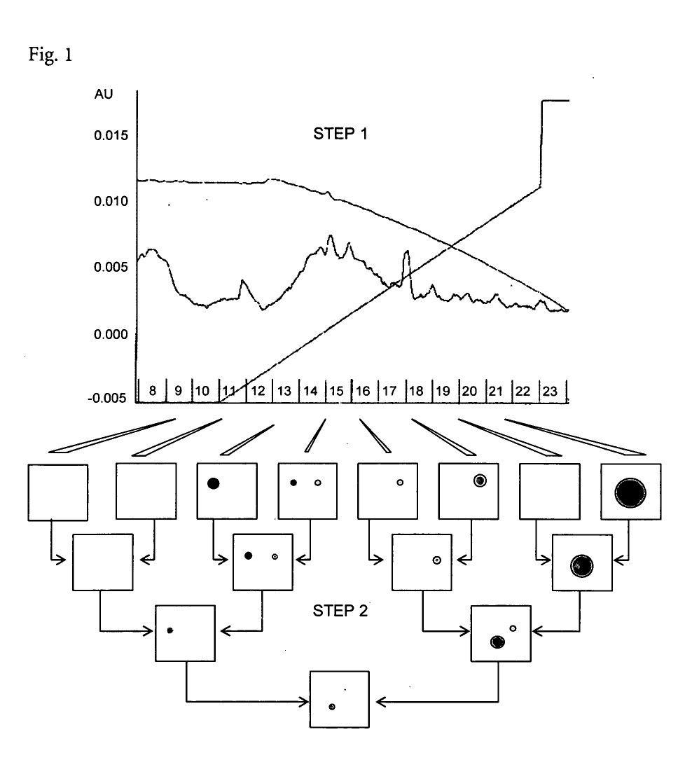

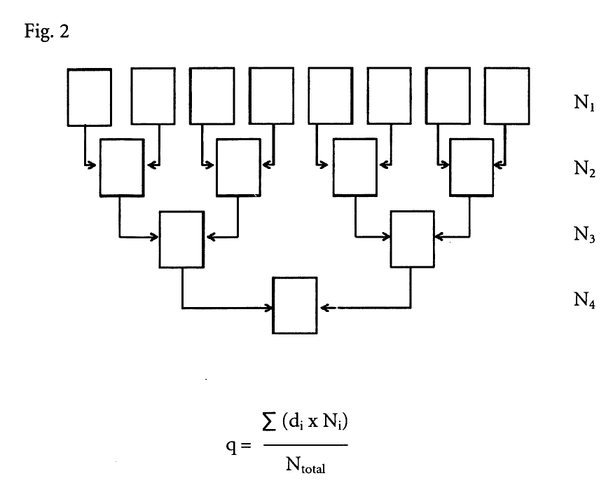

### DETAILED DESCRIPTION OF THE INVENTION

The present invention provides a method for the quantification of a biomolecule in a complex mixture of biomolecules comprising
a. providing at least two fractions derived from the fractionation of the complex mixture of biomolecules comprising each at least one distinct biomolecule component,
b. subjecting the fractions to a serial combinatorial dilution step,
c. detecting and identifying the biomolecule in each original fraction and each diluted fraction by a method with a stable and well defined sensitivity threshold and identity information, and
d. quantifying the biomolecule in the complex mixture of biomolecules by summarizing the number of identifications of the biomolecule in each fraction on each dilution level in consideration of the respective dilution factor.

For the purpose of normalization the sum of d) may be divided by the total number of identifications of all biomolecules in all fractions on all dilution levels (dilution levels of original fractions and diluted fractions).

The method of the present invention for the quantification of a biomolecule provides a relative quantification of one or more biomolecules in a complex mixture of biomolecules from one source compared to the respective biomolecules in a complex mixtures from other sources.

The complex mixture of biomolecules may be derived from any source comprising biological sources comprising cells, cell culture supernatants, biological fluids such as serum, plasma, urine, bronchial lavage fluid, sputum, biopsies like cerebrospinal fluid. The complex mixture of biomolecules comprises at least two different biomolecules. The biomolecule in the present invention may be any biomolecule comprising polynucleotides, polypeptides, proteins, carbohydrates, lipids, glycoproteins, lipoproteins or other modified forms or metabolites thereof. The detection and identification method can be restricted to a single type of biomolecule(s), or can detect and analyze several classes of biomolecules at one time.

The fractionation method used in the method of the present invention should efficiently separate the complex mixture of biomolecules into distinct fractions. Preferably, the complex mixture of biomolecules is fractionated into distinct fractions with each different biomolecule only being present in not more than n minus one fractions wherein n is the total number of fractions and n is equal or higher than two. Preferably, the different biomolecules are present in two different fractions, more preferably in one fraction. The fractionation method which may be used in the method of the present invention may be selected from any method suitable for separation of a complex mixture of the targeted type of biomolecules as known to one of ordinary skill in the art, depending upon the biomolecule to be quantified and each subtype (polypeptide, lipid, etc) of the biomolecule. The fractionation method which may be used in the method of the present invention may be selected from the group comprising fractionation based on adsorption, gravity or sedimentation velocity, electrophoretic fractionation or combinations of these methodologies. For example, in the case of proteins as the target molecule the group includes but is not limited to chromatographic fractionation, ultracentrifugation, protein precipitation, affinity purification, or immunoprecipitation. In the case of peptides (for example obtained from proteolytic digests) as the target molecule the group includes but is not limited to high pressure liquid chromatography (HPLC).

The fractions are then subjected to a serial combinatorial dilution. The serial combinatorial dilution requires at least two fractions to start with. Preferably, the complex mixture of biomolecules is fractionated in as many fractions as necessary to allow a detection and identification of a sufficient number of different targeted biomolecules in the subsequent detection step. Preferably, the number of original fractions is not a prime number, more preferably the number of original fractions is even, and preferably, each initial fraction comprises at least one distinct component.

The number of the fractions to start with the serial combinatorial dilution is dependent on the complexity of the mixture of biomolecules, on the concentration of the individual biomolecules in the complex mixture of biomolecules, the efficiency of the separation methodology, and on the detection and identification method used after the fractionation and the serial combinatorial dilution.

The exact number of dilution steps depends on the sensitivity level that has to be achieved in the experiment. Combining two fractions will dilute the sample twofold, thus limiting the sensitivity of the method to two-fold changes in concentration. Similarly, combining three (N) fractions will limit the sensitivity to three (N-) fold changes.

In conclusion, a reasonable approach to performing the method is to fractionate samples in fraction sizes equal to the resolution of the fractionating methods, and to apply dilutions according to the desired sensitivity. For real world biological samples it rarely makes sense to strive for accuracies larger than two-fold, however, for higher accuracies the present invention permits one of ordinary skill in the art to devise a partial combination/dilution scheme that would yield higher accuracies.

For the serial combinatorial dilution at least two different fractions containing at least one different biomolecule are combined. Preferably, two fractions are combined. This will change the concentration of a biomolecule in the pooled fraction according to the quotient of the dot product of the concentration of the biomolecule in each fraction with the volume of each fraction by the total volume of all combined fractions. In general, this will result in a smaller concentration of any biomolecular component in the diluted fraction compared with the maximum concentration of the respective biomolecule in the original fractions. In the following dilution steps, the concentrations of the individual proteins decrease till they fall below the sensitivity threshold of the subsequent detection and identification method.

The number of dilution steps depends on the starting concentration of the biomolecules in the original fractions, the number of original fractions after fractionation and the detection limit of the detection and identification method.

The method of the present invention further comprises a detection and identification method. The detection method has to feature a defined sensitivity threshold and to provide identity information about the detected biomolecule. Thereby, the presence or absence of a specific biomolecule can be determined in an original fraction or a diluted fraction. The sensitivity threshold does not have to be known in advance for any single species, but must be reproducible for any single species or type of biomolecule. However, the sensitivity threshold itself does not have to be the same for all biomolecules in the sample. The selection of a reproducible sensitivity threshold for any single species of biomolecule, as well as identification method for that biomolecule, is known to those of ordinary skill in the art. The detection and identification method of the present invention may rely on the chemical composition, structure, or sequence of the biomolecule and the physico-chemical or enzymatic properties resulting therefrom. These include hybridization with a specific probe, reaction with a specific antibody or lectin, enzymatic or chemical reaction with a specific molecular probe, isoelectric point, molecular weight, molecular masses of fragments resulting from enzymatic digestion of the biomolecule, NMR spectrum or combinations thereof. For the example of protein quantitation, the detection and identification method of the present invention may be selected from the group comprising combinations of one- or two-dimensional gel electrophoresis with mess spectrometry, immunoassays (e.g. western blot), gas chromatography combined with mess spectrometry (GS/MS) or electrophoresis with specifically labeled molecular entities, e.g. fluorescent, chemical (e.g. biotin), or radioactive tags. The detection and identification method generally does not have a predetermined or known limit of detection, as the only requirement is the reproducibility of the detection at a defined concentration of the biomolecule to be analyzed (analyte).

To derive the quantitation of a biomolecule the identifications or the specific fingerprints (peptide mass fingerprints) of the fractions of each dilution step are calculated whereby the respective dilution factor for each dilution step is considered. The resulting number of identifications of the biomolecule is summarized for all dilution levels. For the purpose of normalization this sum may be divided by the total number of identifications of all biomolecules in all fractions (original fractions and diluted fractions).

$$Relative Quantity ( q ) = \frac{\sum ( {d}_{i} \times {N}_{i} )}{{N}_{total}}$$

wherein Nᵢ is the number N of identifications of an individual biomolecule at dilution level i, dᵢ the dilution factor d of the respective dilution level i and Nₜₒₜₐₗ the total number N of identifications of all biomolecules in all fractions on all dilution levels. Thus, the dilution factor is equal to 1/part of a single fraction in a combined sample, for example, if N neighboring fractions are combined in the sample, then the part of any single fraction is equal to 1 and the dilution fraction is equal to N.

This method is independent of the properties of the various biomolecules. For example, polynucleotides may be processed as well as polypeptides or carbohydrates. A further advantage of this method is that it combines quantification with the identification of a biomolecule in a simple manner without the need for additional efforts targeted at biomolecule quantitation. Moreover, if the quantity of a biomolecule derived of one source shall be compared with the one of another source the mixtures of biomolecules, which comprise one or more biomoecules, may be processed separately of each other. The biomolecule in the present invention may be any biomolecule comprising polynucleotides, polypeptides, proteins, carbohydrates, lipids, glycoproteins, lipoproteins or other modified forms or metabolites thereof.

Having now generally described this invention, the same will become better understood by reference to the specific examples, which are included herein for purpose of illustration only and are not intended to be limiting unless otherwise specified, in connection with the figures, herein described.

The following examples are provided for illustrative purposes and are not intended to limit the scope of applicants' invention.

### EXAMPLES

Commercially available reagents referred to in the examples were used according to manufacturer's instructions unless otherwise indicated.

### Example 1

### Cell Culture

INS-1 cells (Asfari, Janjic et al. 1992) were cultured in RPMI 1640 medium (Invitrogen) supplemented with 10% FCS (Invitrogen, heat inactivated) 10 mM Hepes solution(Invitrogen), 1 mM Na pyruvate (Sigma); 50 μM beta-mercaptoethanol (Sigma), 1% Penicillin/Streptomycin solution (SIGMA), and low (5 mM) or high (10 mM) concentrations of glucose (SIGMA). Cells were generally cultivated at low glucose concentrations. For preparative culture, the cells were split and then incubated in low-glucose medium until cells were confluent. The medium was then changed to either low-glucose or high-glucose medium and incubations were continued for four days. For harvesting, cells were first washed once with Hanks Balanced Salt Solutions (HBSS, Invitrogen) and then covered with Trypsin/EDTA solution for 1-2 min until cells become rounded and detach from the bottle surface. The Trypsin/EDTA solution was discarded and the cells were suspended in Trypsin Inhibitor Solution (SIGMA), transferred to centrifuge tubes and centrifuged at 1200×g for 5 min. After this, the cells were washed three times in HBSS, again using the same centrifuge parameters. The supernatant was aspired and discarded and the pellet was stored frozen at −80620 C. until used for the preparation of cytosol.

### Preparation of Cytosol

All solutions were cooled to 4620 C. (except for HBSS) and all steps were carried out in a cooled environment (ice bath). Ca 10⁸ cells were resuspended in cell homogenization medium (CHM; 150 mM MgCl2, 10 mM KCl, 10 mM Tris, 0.25 M glucose, 1 mM EDTA, pH 7.4) and left on ice for 2 min. The cell suspension was transferred to a Potter-Elvehjem homogenization vessel. The cold pestle of a Potter-Elvehjem homogenizer was attached to an overhead high-torque electric motor and the cells were homogenized using 10 strokes at 1000 rpm. The efficiency of the homogenization (>90% of broken cells) was confirmed by phase-contrast microscopy. Cell debris and nuclei were removed by centrifuging for 5 min at 1000×g. The mitochondria were separated by centrifugation at 5000×g. The enriched cytosolic fraction was finally recovered by centrifuging at 200000×g and by transferring the supernatant to a clean tube. The final protein concentration in the preparation was 2.5-5.0 mg/ml.

### Chromatographic Fractionation

All fractionation steps were carried out using an AKTAexplorer 10 chromatography system (Amersham) at room temperature. The cytosol preparations (10 mg of total protein) were passed through a 0.45 μm Milex-HV syringe-driven filter unit and the loaded onto desalting columns (three 5 ml HiTrap desalting columns connected in series, Amersham). The proteins were eluted using Buffer A (25 mM NaHPO₄⁻ pH 7.5; 1 mM EDTA; 0.5 mM dithioerythritol; 1× Complete EDTA-free (Protease inhibitor cocktail tablets from Roche Diagnostics; pH adjusted to 7.5) using a flow rate of 1.5 ml/min. Proteins were recovered in a 20 ml injectionloop using the increase in UV absorption (280 nm) and the minimum in conductivity as boundaries for the protein fraction. The proteins were then separated by anion exchange chromatography using a TSK DEAE-5PW 7.5 cm×7.5 mm column (TOSOH BIOSEP) at a flow rate of 1 ml/min. Buffer A was used as the binding buffer, buffer B (25 mM NaHPO₄⁻ pH 7.5; 1 mM EDTA; 0.5 mM dithioerythritol; 1× Complete EDTA-free (Protease inhibitor cocktail tablets from Roche Diagnostics; 1 M NaCl, pH adjusted to 7.5) as the elution buffer. The sample was loaded onto the column and unbound material was washed off with 7 column volumes (CV) of Buffer A. The bound proteins were then eluted by three-segment gradient (1^{st} segment: 0-11% Buffer B in 3 CV; 2nd segment: 11-30% Buffer B in 10 CV; 30-50% Buffer B in 1.5 CV). Finally, the column was washed with 5 CV of 50% Buffer B. Fractions of 1 ml were collected and combined to form eight pools plus the flow-through. The conductivity boundaries were: FT: UV280 increase to increase in conductivity; 1 (start of conductivity-increase to 12 mS); 2 (12 to 15 mS); 3 (15 to 18 mS); 4 (18 to 21 mS); 5 (21 to 24 mS); 6 (24 to 27 mS); 7 (27 to 30 mS); 8 (30 to 40 mS).

### Two-Dimensional Electrophoresis

The fractions were concentrated and desalted by reversed phase chromatography using self-packed syringe-driven minicolumns (MoBiTec M1002) filled with 100 mg of POROS 20 R1 material (PerSeptive Biosystems). The columns were washed with 10 ml of 0.1% Trifluoroacetic Acid (TFA) and with 70% Acetonitrile/0.1% TFA. After loading the sample, the columns were washed with 10 ml of 0.1% TFA and eluted with 2 ml of 70% Acetonitrile/0.1% TFA. The eluate was then dried in a SpeedVac evaporator and taken up in IEF Sample Buffer (7 M Urea, 2 M Thiourea, 50 mM Tris pH 7.5, 2 % (w/v) CHAPS, 0.4% (w/v) Dithioerythritol, 0.5% (w/v) ampholytes). Aliquots containing 0.5 mg of protein were set aside from each fraction and labeled as Sample 1 to 8. The following samples were prepared from the remainder of the fractions: Sample 9: 0.25 mg fraction 1+0.25 mg fraction 2; Sample 10: 0.25 mg fraction 3+0.25 mg fraction 4; Sample 11: 0.25 mg fraction 5+0.25 mg fraction 6; Sample 12: 0.25 mg fraction 7+0.25 mg fraction 8; Sample 13: 0.125 mg fraction 1+0.125 mg fraction 2+0.125 mg fraction 3+0.125 mg fraction 4; Sample 14: 0.125 mg fraction 5+0.125 mg fraction 6+0.125 mg fraction 7+0.125 mg fraction 8; Sample 15: 0.0625 mg fraction 1+0.0625 mg fraction 2+0.0625 mg fraction 3+0.0625 mg fraction 4+0.0625 mg fraction 5+0.0625 mg fraction 6+0.0625 mg fraction 7+0.0625 mg fraction 8. Thus, samples 1-8 contain 0,5 mg of protein fractions, samples 9-12 each correspond to a two-fold dilution of these samples, samples 13 and 14 to a four-fold, and sample 15 to an eight-fold dilution of these original fractions. Isoelectric Focusing was performed using immobilized pH gradient (IPG) strips with a pH range from 3 to 10 (IPG 3-10L; Amersham) in a Protean IEF Cell (BioRad) at 20620 C. The dried strips were re-hydrated in a solution containing 7 M Urea, 2M Thiourea, 2 % (w/v) CHAPS, 0.4 % (w/w) Dithioerythritol, and 0.5 % (w/v) ampholytes. The protein fractions were cup-loaded at the cathodic end of the strip. The voltage was linearly increased to 5000V over 8 h, followed by a 5000 V plateau for 10 h. The strips were equilibrated and alkylated by successive washes in Equilibration Solution 1 (6 M Urea, 50 mM Tris pH 7.5, 30 % Glycerol, 2.0 % SDS, 30 mM Dithioerythritol) and Equilibration Solution 11 (6 M Urea, 50 mM Tris pH 8.8, 30% Glycerol, 2.0% SDS, 0.23 M lodoacetamide) for 10 min each. The strips were loaded onto 11% Acrylamide/PDA (37:1) gradient gels (240 mm×200 mm×1.5 mm). The proteins were resolved by electrophoresis at 80V O/N in an ETTAN Dalt Electrophoresis apparatus (Amersham) with constant cooling (20620 C.).

### Gel Staining and Processing

The gels were fixed in 50% methanol/10% acetic acid and stained with Coomassie Blue (Colloidal Blue, Invitrogen, Carlsbad, Calif.) overnight followed by multiple washes in ultra-pure water for 7 h total. The gels were scanned and spots with a diameter of 1.2 mm were excised using an automatic spot picking device. The spots were de-stained in a solution containing 100 mM Ammonium hydrogen carbonate and 30% Acetonitrile. The dried de-stained gel pieces were digested in 5 μl of a 10 μg/ml Trypsin solution (Roche Diagnostics) overnight at room temperature. After addition of 10 μl of ultra-pure water, proteins were extracted with 5 μl of a solution containing 75% Acetonitrile and 0.3% (v/v) TFA. The peptide solution was spotted onto a MALDI target together with α-Cyano-4-hydroxycinnamic acid as matrix.

### Mass Spectrometry and Protein Identification

Peptide masses were measured on a Bruker Ultraflex Instrument (Bruker, Bremen, Germany), using ACTH and Bradykinin as internal mass standards. As explained below, monoisotopic peptide masses were automatically detected from the mass spectra and compared to theoretical masses of peptides derived from an in-silico tryptic digest of all proteins from a database of protein sequences (e.g. SwissProt, or NCBI rat genome draft).

### Peak Annotation for MALDI Mass Spectra

The mass spectrometric data is two times filtered with a low-pass median parametric spline filter in order to determine the instrument baseline. The smoothed residual mean standard deviation from the baseline is used as an estimate of the instrument noise level in the data.

After baseline correction and rescaling of the data in level-over-noise coordinates, the data point with the largest deviation from the baseline is used to seed a non-linear (Levenberg-Marquardt) data fitting procedure to detect possible peptide peaks. Specifically, the fit procedure attempts to produce the best fitting average theoretical peptide isotope distribution parameterized by peak height, resolution, and monoisotopic mass. The convergence to a significant fit is determined in the usual way by tracking sigma values.

After a successful convergence, an estimate for the errors of the determined parameters is produced using a bootstrap procedure using sixteen repeats with a random exchange of ⅓ of the data points.

The resulting fit is subtracted from the data, the noise level in the vicinity of the fit is adjusted to the sum of the extrapolated noise level and the deviation from the peak fit, and the process is iterated to find the next peak as long as a candidate peak more than five times over level of noise can be found. The process is stopped when more than 50 data peaks have been found.

The zero and first order of the time-of flight to mass conversion are corrected using linear extrapolation from detected internal standard peaks, and confidence intervals for the monoisotopic mass values are estimated form the mass accuracies of the peaks and standards.

### Probabilistic Matching of Spectra Peaks to In-Silico Protein Digests

Peak mass lists for mass spectra are directly compared to theoretical digests for whole protein sequence databases. For each theoretical digest, [1-Π(1−N P(pi))]^{cMatches} is calculated, where N is the number of peptides in the digest, P(pi) is the number of peptides that match the confidence interval for the monoisotopic mass of the peak divided by the count of all peptides in the sequence database, and cMatches is the number of matches between digest and mass spectrum. It can be shown that this value is proportional to the probability of obtaining a false positive match between digest and spectrum. Probability values are further filtered for high significance of the spectra peaks that produce the matches. After a first round of identifications, deviations of the identifications for mass spectra acquired under identical conditions are used to correct the second and third order terms of the time-of-flight to mass conversion. The resulting mass values have mostly absolute deviations less than 10 ppm. These mass values are then used for a final round of matching, where all matches having a Pmism less than 0.01/NProteins (1% significance level with Bonferoni correction) are accepted.

### Database Analysis

For each protein in the database, the number of identifications per 2D-PAGE gel analyzed in this study was counted. In this example the dilution level 1 was set as reference. Then, the following values were derived:
Number of identifications in dilution level 1 (undiluted samples, samples 1-8)=N₁
Number of identifications in dilution level 2 (2-fold dilution, samples 9-12)=N₂
Number of identifications in dilution level 3 (4-fold dilution, samples 13,14)=N₃
Number of identifications in dilution level 4 (8-fold dilution, sample 15)=N₄

As expected, for most proteins the N values decreased roughly two-fold from layer to layer. To account for the dilution factors and to derive a rough absolute quantity for each protein, a quantity value q was calculated as follows:
q=(N₁+2×N₂+4×N₃+8×N₄)/total number of identified protein spots for all samples of the same source on all dilution levels

The division by the total number of identification for all samples of the same source was introduced to account for inter-sample variations in protein concentration.

For each protein, the q values for both mixture samples (high and low glucose) were calculated and compared.

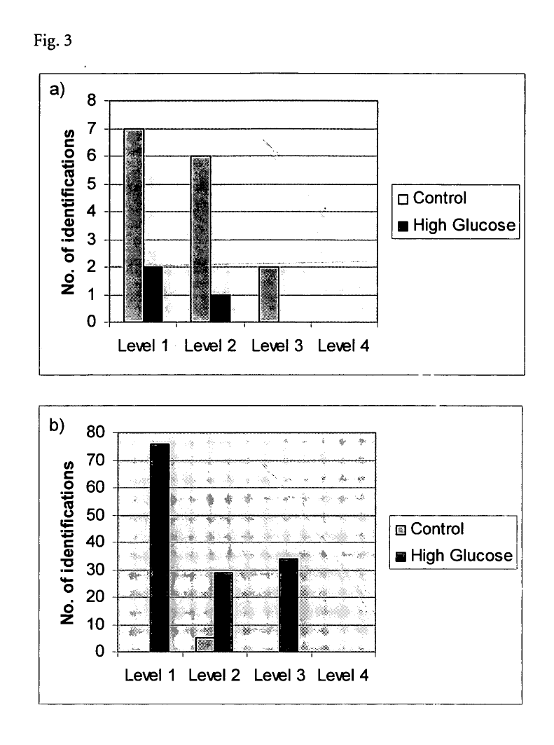

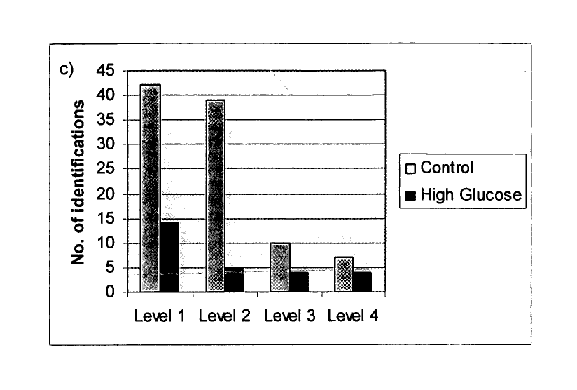

The following three proteins were chosen as examples for the illustration of the feasibility of this quantification method: Glycogen Phosphorylase (liver form); Vimentin, and Heat shock protein 105 (Table 1, FIG. 3).

TABLE 1
relative Quantity (q Values) of the proteins present in the cytosol
obtained for the three experiments for three example proteins

| | q (5 mM Glucose) × 10⁻⁵ | q (10 mM Glucose) × 10⁻⁵ |
|---|---|---|
| Glycogen | | |
| phosphorylase | | |
| Experiment 1 | 112 | 0 |
| Experiment 2 | 8 | 0 |
| Experiment 3 | 124 | 44 |
| Vimentin | | |
| Experiment 1 | 0 | 2130 |
| Experiment 2 | 80 | 305 |
| Experiment 3 | 0 | 1758 |
| Heat shock | | |
| protein 105 | | |
| Experiment 1 | 17 | 13 |
| Experiment 2 | 121 | 39 |
| Experiment 3 | 200 | 89 |

### Example 2

### Collagen Alpha I (IV)

Serum samples from three insulin-resistant and three insulin sensitive patients (Caucasian, female) were fractionated as described below. The Body Mass Index (BMI) and the Glucose Disposal Rate (GDR) as determined by the Euglycemic-Hyperinsulinemic Clamp method (Garvey et al. Diabetes 34 (1985) 222-234) are indicated in Table 2. Combinatorial serial dilution was performed as described in the patent application and the resulting samples were subjected to Two-Dimensional-SDS-Polyacrylamide Gel Electrophoresis (2D-PAGE) as described below. All detectable protein spots were excised from each gel. The proteins were digested with trypsin and the resulting peptides subjected to Matrix-Assisted Laser Desorption Ionization Time-of-Flight Mass Spectrometry (MALDI-MS). Protein identification was achieved by peptide mass fingerprint analysis as described below and protein lists were compared as described in Example 1.

TABLE 2
Body Mass Index (BMI) and Glucose Disposal Rates (GDR)
determined by the euglycemic hyperinsulinemic clamp method of
six subjects. As GDRs above 15 are considered as the breakpoint
for the determination of insulin resistance, the patients on the left side
of the panel are classified as insulin sensitive (IS) and those on the
right side as insulin resistant (IR). Plasma from these individuals was
analyzed by serial combinatorial dilution followed by 2D-PAGE, spot
excision, tryptic digest, MALDI-MS and finally protein identification
by peptide mass fingerprint comparison.

| | |
|---|---|
| Insulin- | Insulin-Resistant |
| Sensitive (IS) | (IR) |

| Patient | BMI | GDR | Patient | BMI | GDR |
|---|---|---|---|---|---|
| IS1 | 22.4 | 21.9 | IR1 | 31.3 | 10.2 |
| IS2 | 22.4 | 19.7 | IR2 | 33 | 11.65 |
| IS3 | 29.5 | 20.4 | IR3 | 33.1 | 8.0 |

### Sample Preparation

A method was established to search for Insulin Resistance markers in human plasma by applying proteomics technologies. Plasma is a difficult to analyze by Proteomics techniques because it includes ca. ten high-abundance proteins, which represent approximately 98% of the total protein mass. The high-abundance proteins, albumin and antibody chains were removed, by applying chromatographic techniques and fractionated the flow through fraction over an ion exchanger. The scheme described comprises three chromatography steps, matrix blue, protein G and ion exchange, and is highly reproducible. All chromatographic steps were performed on an FPLC System (Pharmacia).

### Removal of Albumin by Affinity Chromatography on Mimetic Blue and Removal of Immunoglobulins by Affinity Chromatography on Protein G

Human plasma was received from three control individuals and three patients with diabetes type II. Protease inhibitors cocktail (Roche Diagnostics, Mannheim, Germany) was added to the plasma (one tablet to 50 ml). Plasma was diluted three-fold with 25 mM MES, pH 6.0, to reduce the salt concentration and adjust the pH to about 6.0. The two columns, Mimetic blue SA P6XL (50 ml, ProMetic BioSciences Ltd.) and HiTrap Protein G HP (5 ml, Amersham Biosciences) were connected in series and equilibrated with 25 mM MES, pH 6.0. The volume corresponding to approximately one g of plasma protein(15 ml, 66 mg/ml) was filtered through a 0.22 μm filter and applied onto the Mimetic blue column at 5 ml/min. The flow through of this column was directly loaded onto the Protein G column and the flow-through fraction from the latter column was collected (about 120 mg). The two columns were washed with 100 ml of 25 mM MES, pH 6.0 and then they were separated. The Mimetic blue column was eluted with a step gradient of 2 M NaCl in 50 mM Tris-HCl, pH 7.5 and the Protein G was eluted with 100 mM glycine-HCl, pH 2.8 and the eluate was neutralized with 1 M Tris base. The flow through fraction and the two eluates were analyzed by two-dimensional gels and the proteins were identified by MALDI-MS. In the eluate from Mimetic blue, mainly full-length and fragmented albumin were detected. In the eluate from the Protein G column, mainly heavy and light Ig chains were detected. Most of the other plasma proteins were recovered in the flow through fraction.

### Protein Fractionation by Ion exchange Chromatography

The flow through and the wash fractions from the Mimetic blue and Protein G columns were combined, adjusted to pH 8.0 with 2 M Tris base and were applied onto a HiTrap Q HP column (5 ml, Amersham Biosciences), equilibrated with 50 mM Tris-HCl, pH 8.0 at 5 ml/min. The column was eluted with a liner gradient of increasing salt concentration from 0 to 1 M NaCl in 50 mM Tris-HCl, pH 7.5. Five-ml fractions were collected and analyzed by 1-D gels. Approximately 50 mg of total protein were recovered from this column. On the basis of the gel analysis, the fractions were pooled to form eight pools, so that each pool included about 5 mg of total protein. The pools were concentrated with Ultrafree-15 Centrifugal Filter (5 k MWCO, Millipore) and each of the eight pools was analyzed by 2-D gels. About 400 spots from each gel were excised and analyzed by MALDI-MS.

### 2D-PAGE

Immobilized pH gradient (IPG) strips were purchased from Amersham Biosciences (Uppsala, Sweden). Acrylamide was obtained from Biosolve (Valkenswaard, The Netherlands) and the other reagents for the polyacrylamide gel preparation were from Bio-Rad Laboratories (Hercules, Calif., USA). CHAPS was from Roche Diagnostics (Mannheim, Germany), urea from Applichem (Darmstadt, Germany), thiourea from Fluka (Buchs, Switzerland) and dithioerythritol from Merck (Darmstadt).

Samples of 0.5 mg total protein were applied on 3-10 NL IPG strips, in sample cups at their basic and acidic ends. Focusing started at 200 V, and the voltage was gradually increased to 5000 V at 3 V/min, using a computer-controlled power supply and was kept constant for a further 6 h. The second-dimensional separation was performed either on 12% constant SDS polyacrylamide gels (180×200×1.5 mm) at 40 mA per gel. After protein fixation for 12 h in 40% methanol that contained 5% phosphoric acid, the gels were stained with colloidal Coomassie blue (Novex, San Diego, Calif., USA) for 24 h. Excess dye was washed from the gels with H₂O, and the gels were scanned in an Agfa DUOSCAN densitometer (resolution 400). Electronic images of the gels were recorded with Photoshop (Adobe) software. The images were stored in tiff (about 5 Mbytes/file) and jpeg (about 50 Kbytes/file) formats. The gels were kept at 4° C. until used for MS analysis.

### MALDI-MS

Selected spots of 1.2 mm diameter were excised with a homemade spot picker (described in European Application EP 1 384 994), placed into 96-well microtiter plates and each gel piece was destained with 100 μl of 30% acetonitrile in 50 mM ammonium bicarbonate in a CyBi™-Well apparatus (Cybio AG, Jena, Germany). After destaining, the gel pieces were washed with 100 μl of H₂O for 5 min, and dried in a speedvac evaporator without heating for 45 min. Each dried gel piece was rehydrated with 5 μl of 1 mM ammonium bicarbonate, that contained 50 ng trypsin (Roche Diagnostics, Mannheim, Germany). After 16 h at room temperature, 20 μl of 50% acetonitrile, that contained 0.3% trifluoroacetic acid was added to each gel piece. The gel pieces were incubated for 15 min with constant shaking. A peptide mixture (1.5 μl) was simultaneously applied with 1 μl of matrix solution, that consisted of 0.025% α-cyano-4-hydroxycinnamic acid (Sigma), and that contained the standard peptides des-Arg-bradykinin (Sigma, 20 nM, 904.4681 Da) and adrenocorticotropic hormone fragment 18-39 (Sigma, 20 nM, 2465.1989 Da) in 65% ethanol, 32% acetonitrile, and 0.03% trifluoroacetic acid, to the AnchorChip™. The sample application was performed with a CyBi-Well apparatus. Samples were analyzed in a time-of-flight mass spectrometer (Ultraflex TOF-TOF, Bruker Daltonics) in the reflectron mode. An accelerating voltage of 20 kV was used. Proteins were identified on the basis of peptide-mass matching.

### Peak Annotation for MALDI Mass Spectra

Mass spectrometric data is two times filtered using a low-pass median parametric spline filter in order to determine the instrument baseline. The smoothed residual mean standard deviation from the baseline is used as an estimate of the instrument noise level in the data. After baseline correction and rescaling of the data in level-over-noise coordinates, the data point with the largest deviation from the baseline is used to seed a non-linear (Levenberg-Marquardt) data fitting procedure to detect possible peptide peaks. Specifically, the fit procedure attempts to produce the best fitting average theoretical peptide isotope distribution parameterized by peak height, resolution, and monoisotopic mass. The convergence to a significant fit is determined in the usual way by tracking sigma values. After a successful convergence, an estimate for the errors of the determined parameters is produced using a bootstrap procedure using sixteen repeats with a random exchange of ⅓ of the data points. The resulting fit is subtracted from the data, the noise level in the vicinity of the fit is adjusted to the sum of the extrapolated noise level and the deviation from the peak fit, and the process is iterated to find the next peak as long as a candidate peak more than five times over level of noise can be found. The process is stopped when more than 50 data peaks have been found. The zero and first order of the time-of flight to mass conversion are corrected using linear extrapolation from detected internal standard peaks, and confidence intervals for the monoisotopic mass values are estimated form the mass accuracies of the peaks and standards.

### Probabilistic Matching of Spectra Peaks to In-Solico Protein Digests

Peak mass lists for mass spectra are directly compared to theoretical digests for whole protein sequence databases. For each theoretical digest, [1-Π(1−N P(pi))]^{cMatches} is calculated, where N is the number of peptides in the theoretical digest, P(pi) is the number of peptides that match the confidence interval for the monoisotopic mass of the peak divided by the count of all peptides in the sequence database, and cMatches is the number of matches between digest and mass spectrum. It can be shown that this value is proportional to the probability of obtaining a false positive match between digest and spectrum. Probability values are further filtered for high significance of the spectra peaks that produce the matches. After a first round of identifications, deviations of the identifications for mass spectra acquired under identical conditions are used to correct the second and third order terms of the time-of-flight to mass conversion. The resulting mass values have mostly absolute deviations less than 10 ppm. These mass values are then used for a final round of matching, where all matches having a Pₘᵢₛₘ less than 0.01l/NProteins (1% significance level with Bonferoni correction) are accepted.

### Results

Collagen alpha I (IV) (Collagen IV; Swissprot accession numbers P12109; O00117; O00118; Q14040; Q14041; Q16258) was exclusively detected in two insulin resistant patients (IR2 and IR3, see Table 3). In one patient (IR2), the spots were detected at the second level (two-fold diluted sample), whereas in the second patient (IR3), the protein was detected twice at the forth level (eightfold combinatorial dilution). The number of identifications was multiplied with the dilution factor (in this case, one and four, respectively) and corrected for the total number of protein spots identified for the respective sample.

Collagen IV levels were also measured using an immunoassay (Biotrin Collagen IV EIA; Catalogue Number NoBIO82; Biotrin, Dublin, Ireland) following the supplier's protocol.

The results from the two assays are compared in Table 3.

TABLE 3
Comparison of the results from the serial combinatorial dilution with
the results from the immunoassay.

| Patient | IS1 | IS2 | IS3 | IR1 | IR2 | IR3 |
|---|---|---|---|---|---|---|
| Serial combinatorial dilution | 0 | 0 | 0 | 0 | 10 | 1 |
| Collagen IV EIA (ng/ml) | 108 | 111 | 139 | 86 | 208 | 158 |

IS = Insulin-sensitive patient,
IR = Insulin-resistant patient.
Serial combinatorial dilution: The number of identifications were adjusted for dilution factor and total spot count.
Immunoassay (Collagen IV EIA): The Collagen IV levels were determined by the Biotrin Collagen IV EIA was used. The presented results are the mean of duplicate measurements.

The limit of detection of the described proteomic methodology lies above that for the immunoassay at approx. 50 ng/ml. Above that level, proteins can be detected and coarsely quantified by serial combinatorial dilution coupled to the described identification method. Although no absolute quantification is observed, there is some rank correlation, i.e. the samples with the highest and second highest levels were correctly identified.

The serial combinatorial dilution method of the present invention provides an easy and inexpensive method for the quantitation of a biomolecule. For example, the method of the current invention is an efficient tool to quantify hundreds of proteins in parallel and to identify proteins (eg via Proteomics type large scale protein identification) with marked differences in concentration which can be used in differential protein expression analysis, e.g. for biomarker identification studies. Those skilled in the art will appreciate the scope and breadth of the present invention for the quantitation of a biomolecule. Although preferred embodiments of the invention have been described using specific terms, such description is for illustrative purposes only, and it is to be understood that changes and variations may be made without departing from the spirit or scope of the following claims.

1. A method for the quantification of a biomolecule in a complex mixture of biomolecules comprising
a) providing at least two fractions of a fractionation of a mixture of biomolecules comprising each at least one distinct component, wherein the at least two fractions are separated by ultracentritugation, protein precipitation, or immunoprecipitation,
b) subjecting the fractions to a serial combinatorial dilution,
c) detecting and identifying the biomolecule in each original fraction and each diluted fraction by a detecting and identifying method providing a sensitivity threshold and identity information, wherein the detecting and identifying method comprises one or more of the group consisting of two dimensional gel electrophoresis, mass spectrometry, immunoassays, gas chromatography or electrophroesis with specifically labeled molecular entities,
d) quantifying the biomolecule in the complex mixture by summarizing the number of identifications of the biomolecule in each fraction on each dilution level in consideration of the respective dilution factor.

2. The method of claim 1, wherein the biomolecule is selected from the group consisting of polypeptides, polynucleotides, proteins, carbohydrates, lipids, glycoproteins, lipoproteins or metabolites thereof.

3. The method of claim 1 wherein the biomolecule is present in not more than n−1 fractions wherein n is the total number of fractions and wherein n is equal or higher than two.

4. The method of claim 1 wherein the summarizing step of quantifying step d) is divided by the total number of identifications of all biomolecules in all fractions on all dilution levels, according to the equation
$$Relative Quantity ( q ) = \frac{\sum ( {d}_{i} \times {N}_{i} )}{{N}_{total}}$$
wherein Nᵢ is the number N of identifications of an individual biomolecule at dilution level i, dᵢ is the dilution factor d of the respective dilution level i and Nₜₒₜₐₗ is the total number N of identifications of all biomolecules in all fractions on all dilution levels.

5. The method of claim 3 wherein the biomolecule is present in two fractions.

6. The method of claim 3 wherein the biomolecule is present in one fraction.

7. A method for the quantification of a polypeptide or protein in a complex mixture of biomolecules comprising
a) providing at least two fractions of a fractionation of a mixture of biomolecules comprising each at least one distinct polypeptide or protein, wherein the at least two fractions are separated by ultracentrifugation, protein precipitation, or immunoprecipitation,
b) subjecting the fractions to a serial combinatorial dilution,
c) detecting and identifying the polypeptide or protein in each original fraction and each diluted fraction by a detecting and identifying method providing a sensitivity threshold and identity information, wherein the detecting and identifying method comprises one or more of the group consisting of two dimensional gel electrophoresis, mass spectrometry, immunoassays, gas chromatography or electrophroesis with specifically labeled molecular entities,
d) quantifying the polypeptide or protein in the complex mixture by summarizing the number of identifications of the polypeptide or protein in each fraction on each dilution level in consideration of the respective dilution factor.

8. The method of claim 1 wherein the polypeptide or protein is present in not more than n−1 fractions wherein n is the total number of fractions and wherein n is equal or higher than two.
